(19) United States
(12) Patent Application Publication
(10) Pub. No.: US 2008/0293091 A1
(43) Pub. Date: Nov. 27, 2008

(54) APPARATUS AND METHODS FOR AUTOMATED DIFFUSION FILTRATION, CULTURING AND PHOTOMETRIC DETECTION AND ENUMERATION OF MICROBIOLOGICAL PARAMETERS IN FLUID SAMPLES

(76) Inventors: Ravi Kanipayor, London (CA); Ron Emburgh, Mississauga (CA)

Correspondence Address: BERESKIN AND PARR, 40 KING STREET WEST, BOX 401, TORONTO, ON, M5H 3Y2, CA

(21) Appl. No.: 12/126,366

(22) Filed: May 23, 2008

Related U.S. Application Data
(60) Provisional application No. 60/940,214, filed on May 25, 2007.

(51) Int. Cl. C12Q 1/02; C12M 1/34; C12M 1/36; C12M 1/00

(52) U.S. Cl. 435/29; 435/288.7; 435/286.1; 435/287.3

(57) ABSTRACT

A system providing non-intrusive, automated culturing and photometric detection for analyzing microbiological parameters is described. The system includes a sealed non-intrusive sample cuvette, a housing with an enclosable cover, systems for incubation and photometric detection mounted within the housing. The sample cuvette consists of a clear graduated optically transmitive container with two chambers—a culture chamber and a detection chamber, separated by a permeable membrane wall. The cuvette has an upper part and a lower part of different dimensions. The upper part is bigger in size than the lower part. The sealed top of the cuvette has two fluid inlet/outlet ports for the introduction of the sample into one chamber while when connected to a suitable vacuum device, the second chamber receives the filtered sample through the permeable membrane. The housing has a cuvette holder that is shaped to provide a very snug fit for the cuvette. When placed inside the cuvette holder the bottom of the upper part of the cuvette rests on top of the holder while the bottom part snugly fits inside the holder cavity. The housing with the enclosable cover provides a thermal chamber during simultaneous incubation and photometric enumeration of microbiological parameters. The cuvette holder accommodates a heating element and a temperature sensor. Photometric detection components comprising LEDs and detectors are placed strategically within the holder.

### FIELD OF THE INVENTION

This invention relates to methods and apparatus for the rapid photometric detection and enumeration of microbiological materials in fluid samples.

### BACKGROUND OF THE INVENTION

The membrane filtration method is the standard for testing microbiological parameters in water samples. One advantage of the standard membrane filtration method is that it separates the organism under investigation from the potential toxic components present in the original sample matrix. However, the standard membrane filtration method has several drawbacks. The method is over 200 years old and no major modifications have been done to improve the methodology. It is very imprecise, and has problems in dealing with samples containing high microbial counts. The standard membrane filtration method requires manual, visual counting of the growth on the filter as well as experience in identifying false growth. The method is highly error prone due to uncertainty in capturing the target organism within the measurable range.

The development of chromogenic/fluorogenic reagents has opened the door to methods for the analysis of microbiological materials in fluid samples, using optical spectroscopy. These photometric methods are very sensitive, and can detect the presence of a very low concentration of color producing components of interest in fluid samples (in parts per million), whereas the human eye can only detect the color when these components are present in very high concentration. However, most spectrophotomeric methods are performed outside the culture chamber by drawing an aliquot of a sample from the incubation vessel to photometric tubes at various intervals and measuring using standard spectrometers. This is not only time consuming but requires separate incubators and spectrometers, and technical personnel to conduct the tests, using robotic sampling systems in some cases. There is also a potential risk of cross contamination and human error, if proper care is not applied in conducting the analysis.

The present inventors have developed a photometric apparatus and method for the rapid analysis of microbiological materials, which is the subject of U.S. patent application Ser. No. 11/122,089, published under publication No. US2005/0266516 on 1 Dec. 2005. This apparatus comprises a specimen container made from an optically transparent material, a housing having a container holder mounted within an incubation chamber shaped for holding the specimen container, incubation components mounted within the container holder for incubating microbiological materials within the sample, and spectrophotometer components for measuring the light absorbed, emitted or scattered by the liquid as the microbiological materials are incubated over time. This apparatus and method are superior to standard membrane filtration methods, in that they provide for a rapid but simple, reliable and accurate on site testing of microbiological materials in various samples. This apparatus and method also provide a very large linear dynamic range (>10⁵) without the need for dilution. However, this apparatus is not suitable for use in testing some samples, such as samples containing components that are toxic to the microbiological organism under investigation, and samples containing high suspended solids that can interfere with optical measurements.

There accordingly remains a need for further apparatus and methods for the rapid photometric detection and enumeration of microbiological materials, having advantages associated with both the standard filtration method and photometric methods.

### SUMMARY OF THE INVENTION

The present invention encompasses an apparatus and methods for non-intrusive, automated culturing, and simultaneous rapid photometric detection and quantitation of microbiological, biological, chemical and toxic materials in fluid samples through a continuous, bi-directional diffusion filtration.

The apparatus of the present invention includes a sealed non-intrusive sample cuvette, a housing comprising a base unit with a removable lid, and systems for incubation and photometric detection mounted within the housing. The sample cuvette consists of a clear graduated optically transparent container with two chambers—a culture chamber and a detection chamber—separated by a permeable membrane wall. The cuvette has an upper part and a lower part. The upper part is of different size than the lower part. The sealed top of the cuvette has two fluid inlet/outlet ports for the introduction of the sample into one chamber, while when connected to a suitable vacuum device, the second chamber receives the filtered sample through the permeable membrane. The base of the culture chamber of the cuvette may have an upward indentation to accommodate the temperature sensor cavity of the holder.

The housing has a cuvette holder that is shaped to provide a very snug fit for the cuvette. When placed inside the cuvette holder, the bottom of the upper part of the cuvette rests on top of the holder while the bottom part snugly fits inside the holder. The housing with the cover provides a thermal chamber during simultaneous incubation and photometric enumeration of microbiological parameters. The cuvette holder accommodates a heating element and a temperature sensor. The base of the holder may have an upwardly extending finger cavity to accommodate a temperature sensor. Photometric detection system components, comprising LEDs and detectors, are placed strategically within the holder.

The sealed specimen cuvette acts as a non-intrusive filter apparatus, a specimen culturing vessel as well as an optical sample cuvette. By connecting the outlet from the detection chamber to a vacuum pump, fluid samples can be conveniently drawn into the culture chamber and through the permeable membrane into the detection chamber until the fluid level reaches the volume mark level in the cuvette. The fluid portion in the detection chamber is filtered according to the pore size of the membrane. The pore size can be suitably selected to retain the microbes under investigation in the culture chamber. The membrane is permeable and thus allows movement of fluid sample along with dissolved components in both directions. By connecting both outlets to a bidirectional pump the direction and rate of the flow can be controlled. As an alternative, a closed circulation loop can be created by connecting both outlets through a peristaltic pump or other suitable devices. This way fluid samples from detection chamber can be drawn back into the culture chamber thus providing homogenization of the dissolved components in both chambers at any given time.

The specimen cuvette is made of material that allows the propagation of light. The cuvette may be completely cylindrical or square, with planar vertical surfaces, on opposite sides. Flat, planar surfaces reduces light scattering when using photometric detection system.

In one aspect, the present invention provides a method whereby a fluid sample is introduced into the sterile sealed cuvette through the inlet of the culture chamber using a suction filtration apparatus such as a vacuum pump connected to the outlet of the detection chamber. The culture chamber can be pre-loaded with the appropriate amount of the chromogenic/flurogenic reagent specific to the microbe under investigation or can be introduced as a reagent fluid through the inlet. The fluid sample first enters the culture chamber where the soluble reagent is mixed with it. The fluid sample with the dissolved reagent is then passed through the permeable membrane into the detection chamber. The pores of the membrane is such that it prevents the migration of the microbe under investigation into the detection chamber. The detection chamber receives only the fluid samples and dissolved components which are smaller than the pore size. The fluid is drawn until the level of the fluid reaches the test volume mark in both chambers. A soluble optically active ingredient within the reagent provides the detectable parameter such as color, fluorescence, turbidity, chemiluminescence etc. The detectable parameter provides the detectable signal. The incubation and the detection process are initiated by placing the cuvette directly inside a cuvette holder of the apparatus, placing the removable cap on the apparatus base and initiating the incubation/detection cycle. The detectable signal can be automatically monitored and recorded with time using a computer system and custom software.

In a second aspect, the present invention provides a method whereby a fluid sample is introduced into the sterile sealed cuvette through the inlet of the culture chamber using a suction filtration apparatus such as a vacuum pump connected to the outlet of the detection chamber. The samples are drawn through the inlet of the culture chamber and discarded through the outlet of the detection chamber until the desired volume of the sample is filtered. The permeable diffusion membrane pore size is such that it retains the microbe on the culture chamber and discarding the original sample matrix. Once the desired volume of sample is filtered, the inlet of the culture chamber is connected to a reservoir containing pre-prepared sterile water containing the appropriate chromogenic/flurogenic reagent. By activating the vacuum pump, this reagent solution is then introduced into both chambers until the level of the solution reaches the mark in both chambers. A soluble optically active ingredient within the reagent provides the detectable parameter such as color, fluorescence, turbidity, chemiluminescence etc. The detectable parameter provides the detectable signal. The incubation and the detection process are initiated by placing the cuvette directly inside a cuvette holder of the apparatus, placing the removable cap on the apparatus base and initiating the incubation/detection cycle. The detectable signal can be automatically monitored and recorded with time using a computer system and the custom software.

The detectable time is measured and is correlated to the initial population of the microbe under investigation. Alternately, the apparatus can be left alone to complete the analysis and the data can then be downloaded into the computer using custom software and hardware.

### BRIEF DESCRIPTION OF THE DRAWINGS

The invention will now be described, by way of example only, with reference to the following drawings, in which:

FIG. 1 is an exploded perspective view of apparatus made in accordance with an embodiment of the present invention, showing the cap off and the sample cuvette removed from the base unit;

FIG. 2 is a perspective view of the subject apparatus, showing the sample cuvette placed in the cuvetter holder, with the cap shown in dotted outline;

FIG. 3 is a perspective view of the sample cuvette of the present invention;

FIG. 4 is a sectional side view of the sample cuvette showing a fluid sample inside the cuvette, and a vacuum pump mounted on the top of the sample cuvette;

FIG. 5 is a top view of the base unit showing the cuvette holder;

FIG. 6 is a sectional view of the base unit with the sample cuvette placed in the cuvette holder, taken along line 6-6 in FIG. 5; and

FIG. 7 is a top view of an apparatus made in accordance with an alternative embodiment of the present invention, having multiple, independent cuvette holders with incubation and optical detection blocks within a single base unit.

### DETAILED DESCRIPTION OF THE INVENTION

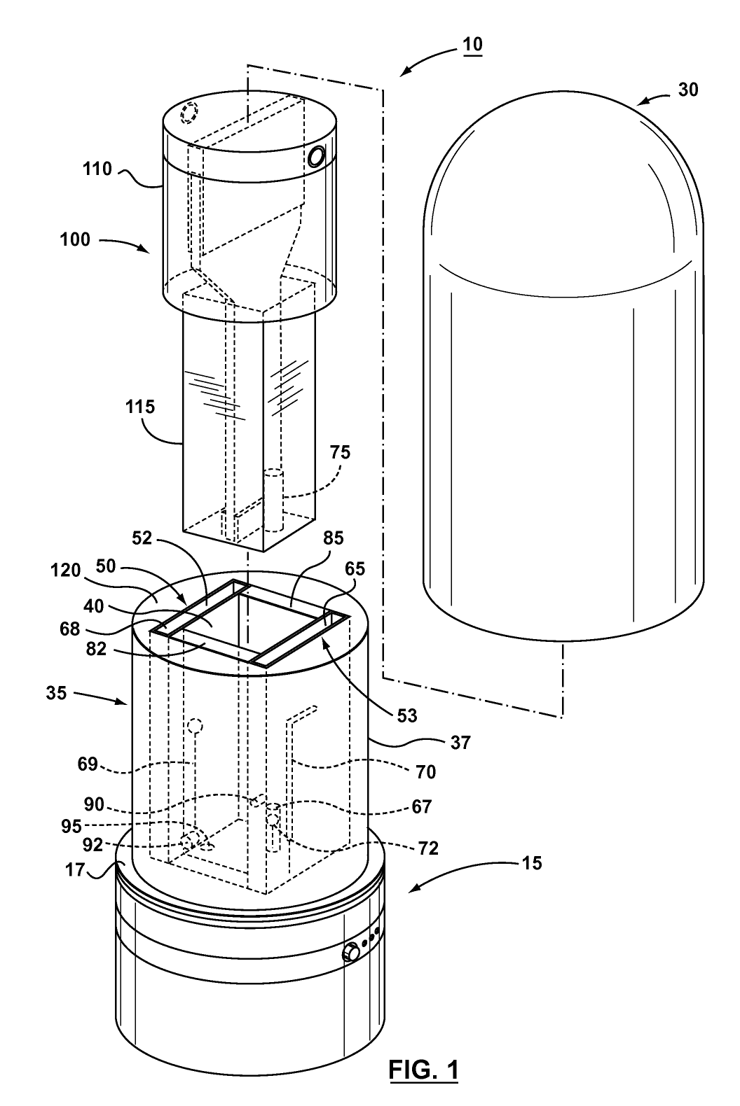

Referring now to FIGS. 1-6, illustrated therein is an apparatus 10 for photometric detection of microbiological parameters, made in accordance with an embodiment of the present invention. Apparatus 10 comprises a sample cuvette 100, a housing comprising a base unit 15, a cuvette holder 35 mounted on the base unit 15, and a removable lid 30 shaped for enclosing the sample cuvette 100 and the cuvette holder 35. The base unit 15 is generally cylindrical and the cuvette holder 35 extends upwardly from the middle of the top surface 17 of the base unit 15. The cuvette holder 35 has a generally cylindrical outside surface 37, and is provided with a centrally located sample cavity 40 shaped to accommodate the sample cuvette 100. The dimensions of the sample cavity 40 are such that when the sample cuvette 100 is placed in the cuvette holder 35, the sample cavity 40 provides a very snug fit for enhanced photometric detection.

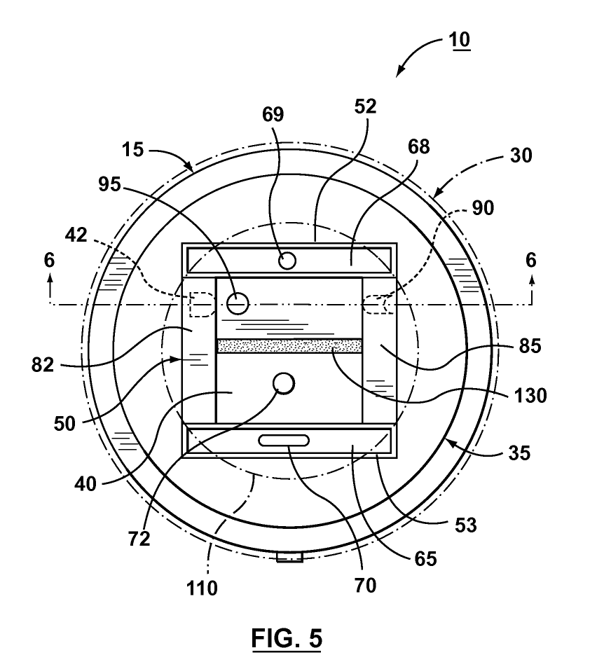

As best shown in FIGS. 1 and 5, sample cavity 40 is generally square, and is defined by square block portion 50 of cuvette holder 35. Square block portion 50 comprises a pair of opposed, hollow sidewall blocks 52, 53 that define chambers shaped to accommodate the temperature control components 69, 70, and a pair of generally solid sidewall blocks 82, 85 that hold the spectrometer components 90, 92.

The hollow sidewall block 53 defines the heating element chamber 65. An indentation at the bottom of the cuvette holder 35 defines the temperature controller cavity 67. Optionally, the opposed side wall block 52 may define the temperature controller chamber 68 to accommodate a second sensor 69 for the optical chamber. The heating element chamber 65 accommodates a heating element 70 while temperature controller cavity 67 accommodates a temperature sensor 72. The heating element 70 and the temperature sensors 69, 72 are controlled by a temperature controller located in base unit 15.

The generally solid sidewall block 85 acts as an LED block while the opposite sidewall block 82 acts as a detector block. The LED block 85 accommodates a light emitting source such as LED 90 placed strategically facing the cavity 40. The detector block 82 accommodates a suitable detector 92, to receive the light emitted from the LED 90 and propagated through the sample cuvette 100. Another suitable LED 95 is placed strategically at the bottom of the cuvette holder 35 near the detector block 82 such that the light emitted from this LED 95 propagates upwardly into the sample cuvette 100. The detector 92 also receives the light emitted by the sample in the cuvette 100 when the sample is exposed to the light scattered from LED 95. The LEDs 90, 95 and the detector 92 are controlled by a microprocessor controller located in the base unit 15. The microprocessor controller processes the signals from the detector 92 and generates a record of the signals as a function of time.

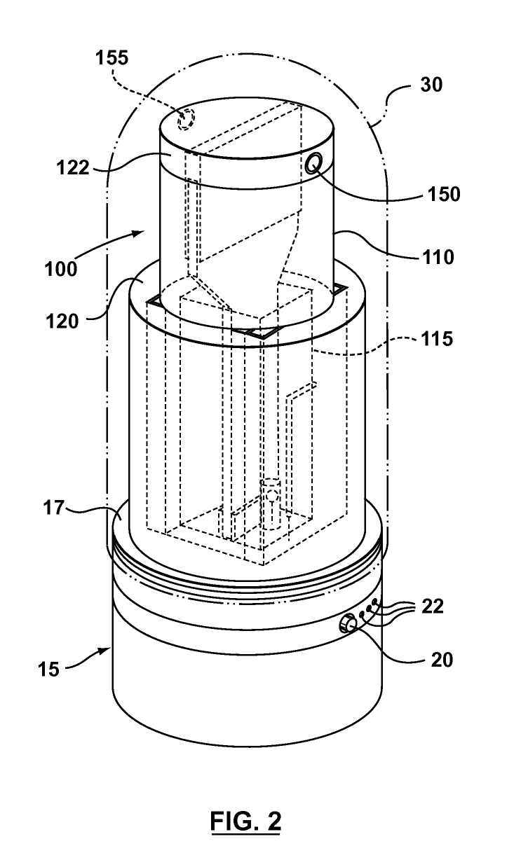

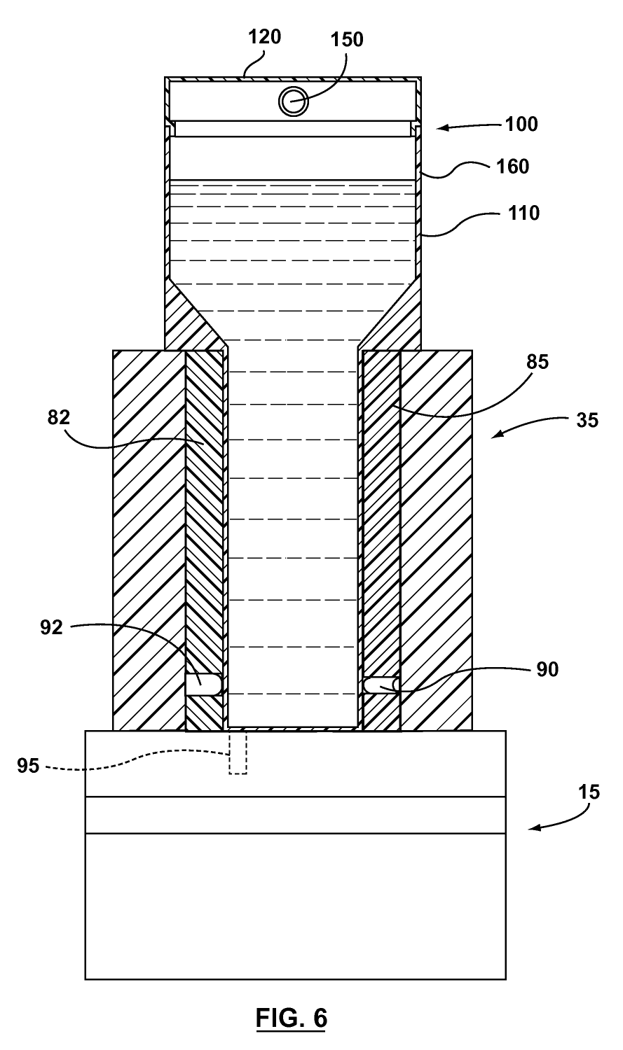

The specimen cuvette 100 comprises a generally cylindrical upper portion 110 having a round cross-section, and a bottom portion 115 having a generally square cross-section. As shown in FIGS. 2 and 6, when the specimen cuvette 100 is placed inside the cuvette holder 35, the sample cavity 40 provides a snug fit between the lower portion 115 of the cuvette 100, while the upper portion 110 sits firmly on top 120 of the round cuvette holder 35 to prevent stray light entering into the cavity 40 and the cuvette 100. The sample cuvette 100 includes a top portion 122 that is sealed to the upper portion 110. The top portion 122 has two inlet/outlet ports 150, 155 that allow fluid to flow into and out of the cuvette 100.

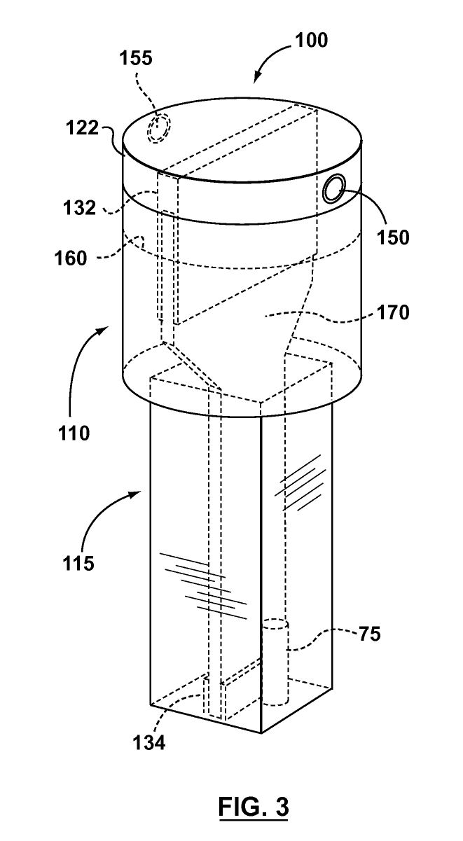

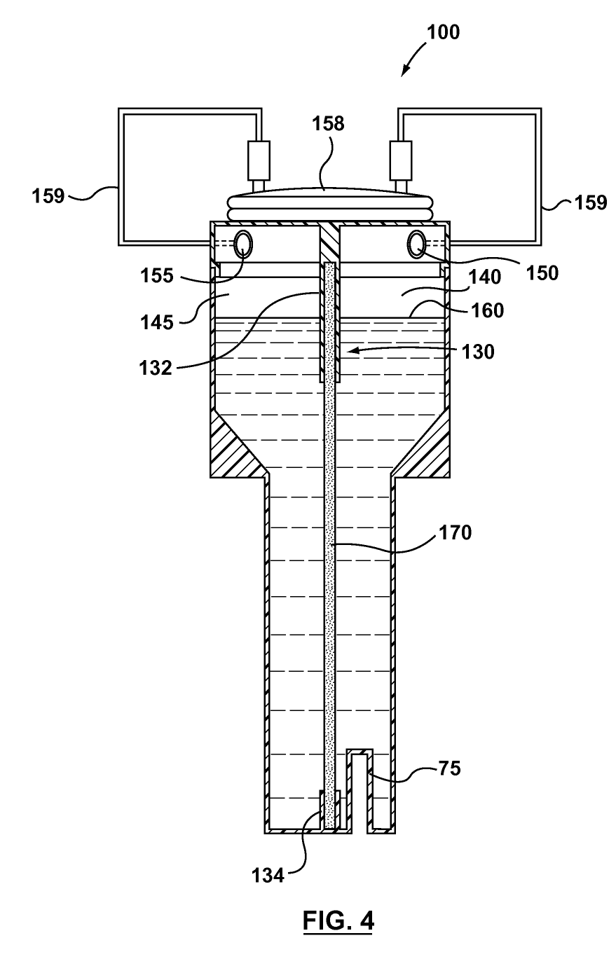

As best shown in FIGS. 3 and 4, the inside of the sample cuvette 100 has two chambers 140, 145 separated by a suitable permeable diffusion membrane cartridge 130. Chamber 140 is the culture chamber and chamber 145 is the detection chamber. The membrane cartridge 130 is attached to the inside of the cuvette 100 by mounts 132, 134, and is firmly sealed on the top and bottom to provide a leak proof system. The cuvette 100 has a distinct calibrated volume marking 160 that provides the exact volume as per the mark when filled with fluid up to the level of the marking 160.

The bottom of the culture chamber 140 has an upwardly projecting indentation 75 to accommodate the sensor cavity 67 when the cuvette 100 is placed inside the sample cavity 40.

The permeable membrane 170 of the membrane cartridge 130 is made of suitable material with a specific pore size to allow only the fluid and the dissolved components to diffuse back and forth between the two chambers. The microbes under investigation and other non-specific particles are blocked by the membane 170 and retained in the culture chamber 140, while the fluid and the parameters attached to enzymes produced by the microbes when subjected to the reagent pass through the membrane 170 into the detection chamber 145. Examples of suitable permeable membrane materials include mixed cellulose, ester, polycarbonate, Millipore® Durapore PVDF.

The culture chamber 140 has an inlet 150 to allow the introduction of the test sample and the detection chamber 145 has an outlet 155, which when connected to a suitable vacuum pump, such as nano pump 158 shown placed on top of cuvette 100 in FIG. 4, can draw samples through the inlet 150 into the culture chamber 140 and through the permeable diffusion membrane 170 into the detection chamber 145. The amount of fluid drawn is controlled by the nano pump 158 or other suction filtration mechanism, and is usually stopped when the fluid reaches the pre-set calibrated marking 160. The nano pump 158 is connected to inlet/outlet ports 150, 155 by hoses 159, and may be powered by an electrical connection to base unit 15.

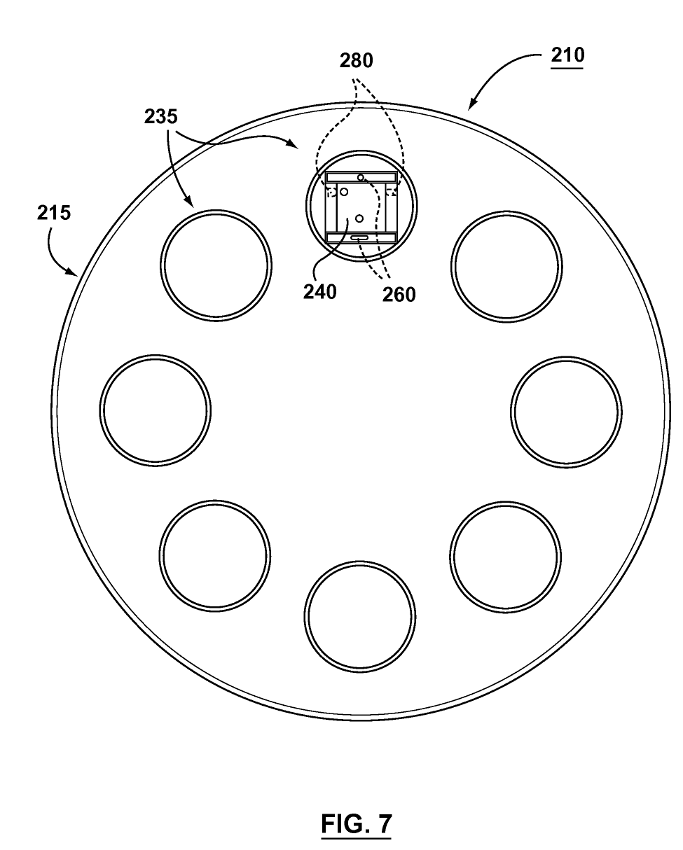

Referring now to FIG. 7, illustrated therein is an apparatus 210 made in accordance with an alternative embodiment of the present invention. Apparatus 210 is similar to the apparatus 10 as shown in FIG. 1, with the exception that apparatus 210 comprises multiple cuvette holders 235 residing within a single base unit 215.

The base unit 215 is generally similar to base unit 15, except base unit 215 is much larger and is shaped to accommodate multiple, independent cuvette holders 235. Each cuvette holder 235 contains a sample cavity 240, incubation components 260 and optical detection components 280. The sample cavity 240 receives the sample cuvette 100 as shown in FIG. 3. Each cuvette holder 235 is independently controlled and monitored for incubation and photometric detection.

TEST METHOD—In a preferred sample cuvette such as that shown in FIG. 4, the outlet 155 is connected to a vacuum pump such as nano pump 158, and the fluid is drawn through the inlet 150 aseptically into the culture chamber 140 and through the permeable membrane cartridge 170 into the detection chamber 145. Only the fluid and the dissolved components are drawn into the detection chamber 145 while the microbes and suspended particles are retained in the culture chamber 140. The fluid is drawn until the level of the fluid in both chambers reached the pre-defined volume mark 160. The outlet 155 is detached from the pump and connected to the inlet 150 through a micro peristaltic pump or other suitable devices.

To provide homogenization of fluid and dissolved components in both chambers at any given time during the incubation and photometric detection, the pump is activated at a pre-set frequency to draw fluid sample from detection chamber into culturing chamber and through the permeable membrane back into detection chamber. As an alternative homogenization of the fluid in the two chambers can be achieved through a diffusion of fluid through the permeable membrane in both directions using a reversible pump.

For simultaneous testing of total coliform and e.coli in water samples, typical reagent solution provides not only nutrient for the growth but also colorimetric or fluorometric detection signals from the growth of total coliform and e.coli respectively. Examples of typical chromogenic/fluorogenic reagents for total coliform and e.coli are Merck KGaA—Readycult Coliform 100®, IDEXX—Colilert®, and CPI-Colitag™.

The reagent is either pre deposited in the incubation chamber 140 of the sealed cuvette 100 or can be added along with the fluid drawn through the inlet 150. After adding portion of the fluid or fluid-reagent solution, gently shake several times the specimen cuvette 100 until the reagent is completely dissolved in the fluid. Draw the remaining fluid until the level of the fluid reached the mark 160 in both chambers.

The sample cuvette 100 can also be used as a sampling vessel whereby samples are drawn in through the inlet 150 and drawn out through the outlet 155 using a suitable vacuum pump attached to the outlet 155. This way large amount of samples (>100 mis) can be analyzed without the need of dilution and eliminate the toxic effect of the matrix on the microbes during culturing. The reagent and sterile water for culturing can be then introduced into both culture chamber 140 and detection chamber 145 through the diffusion filtration process.

Referring now to FIG. 2, for testing of the microbes, the specimen cuvette 100 is placed inside the cavity 40 of the preferred embodiment, and the lid 30 is placed on top of the base unit 15 and twisted tight to provide a seal. The power button 20 is pressed to initiate the test. The temperature controller and microprocessor controller within the base unit 15 are activated, and the test progress is monitored through the operational LEDs (green-yellow-red) 22. At the end of the test, indicated by the LEDs 22, the base unit 15 is connected to a computer and the data is transferred to custom software, such as that described in the inventors' U.S. patent application Ser. No. 11/122,089, the disclosure of which is incorporated herein by reference. The custom software provides the appropriate test results.

It will be apparent that the present system and method combines membrane filtration with optical photometric detection, and that this combination provides several advantages. Removing the microbes from the toxic medium prior to testing provides more consistent results when analyzing various types of matrices. Secondly, accuracy and precision can be improved by filtering volumes of samples greater than 100 mis (standard reporting volume). In the standard membrane filtration method it is not possible to filter more sample because of the limitation of growing microbes on a small filter and the error in reading them, when present in high numbers.

The present system provides for the rapid but simple, reliable and accurate onsite testing of biological, microbiological and chemical parameters in drinking water, recreational water, wastewater, food, medical and environmental samples to provide a better management of production, treatment and other facilities to protect public health and the environment. By combining membrane filtration and optical detection method, higher speed, accuracy and precision can be achieved, and it is believed that apparatus of the present invention should be of great value to those to conduct routine microbiological testing whether in the lab or in remote areas.

The advantages of the apparatus and methods in accordance with the present invention include the following:
1) Large sample volume (>1 Litre) can be used to improve accuracy and precision.
2) The microbes can be cultured under ideal, non-toxic matrix to increase resuscitation of stressed microbes and to prevent non-culturability of viable microbes.
3) Light scattering interference from optical measurements is eliminated.
4) Clogging of the membrane during filtration of high suspended solid sample is eliminated.
5) Difficulty in enumerating microbial colonies on membrane filter with high suspended solids is eliminated.

It is to be understood that the present invention is not to be limited in scope by the specific embodiments, methods or applications as described herein. The above descriptions are intended as an illustration of one aspect of the invention and any modification or alternative embodiments are within the scope of the invention and will be apparent to those skilled in the art. For example the embodiment described herein can be applied for detection and enumeration of parameters biological, microbiological and chemical in nature and the method can be colorimetric, fluorometric, turbidimetric, chemiluminescence or bioluminescence. It should be also appreciated that the scope of the present invention is not limited from DNA and semiconductor-nano-technology based monitoring methods. Also, the cuvette can be “U” shaped with the filter cartridge sitting at the middle of the bottom curve to provide two separate chambers, or inner and outer containers providing two chambers with the walls of the inner chamber made of diffusion membrane can also be envisaged.

Accordingly, various modifications can be made to the embodiments of the invention described and illustrated herein without departing from the present invention, the scope of which is defined in the appended claims.

1. Apparatus for analysis of microbiological parameters in a fluid sample, comprising:
(a) a sample cuvette having two chambers separated by a permeable membrane, each of the chambers having an inlet/outlet port for introduction, filtration and diffusion of fluid within the chambers;
(b) a housing comprising a base unit, a cuvette holder mounted on the base unit, the cuvette holder being shaped to accommodate the sample cuvette, and a cap for enclosing the holder and the cuvette;
(c) temperature control components mounted within the housing proximate the cuvette holder for microbiological culturing; and
(d) spectrophotometer components mounted within the housing proximate the cuvette holder for photometric detection of the microbiological parameters.

2. The apparatus defined in claim 1, wherein the sample cuvette is made of a material that allows for the propagation of light therein.

3. The apparatus defined in claim 1, wherein the permeable membrane has a pore size selected to block microbes under investigation from passing therethrough.

4. The apparatus defined in claim 1, wherein the two chambers comprise a culture chamber and a detection chamber.

5. The apparatus defined in claim 4, wherein the spectrometer components comprise a light source positioned to propagate light within the detection chamber and a light detector positioned to detect the light from the light source that has propagated or been scattered within the detector chamber.

6. The apparatus defined in claim 5, wherein the base unit comprises a microprocessor controller for controlling the light source and the light detector and for processing signals from the light detector and for generating a record of the signals as a function of time.

7. The apparatus defined in claim 4, wherein the temperature control components comprise a heating element and a temperature sensor mounted in the cuvette holder proximate the culture chamber.

8. The apparatus defined in claim 7, wherein the base unit comprises a heating controller for controlling the heating element in response to the temperature sensor.

9. The apparatus of claim 1, further comprising a pump for pumping the fluid from one of the chambers through the membrane into the other of the chambers.

10. The apparatus defined in claim 1, wherein the cuvette holder has a cavity shaped to accommodate a bottom portion of the sample cuvette.

11. The apparatus defined in claim 10, wherein the bottom portion of the sample cuvette is a generally square in cross section and the cavity in the holder is generally square in cross section.

12. The apparatus defined in claim 11, wherein the bottom portion of the sample cuvette has flat, planar opposed sides.

13. The apparatus of claim 11, wherein the cuvette holder comprises a pair of opposed hollow block portions shaped for accommodating the heating element.

14. The apparatus of claim 11, wherein the cuvette holder comprises a pair of opposed generally solid block portions, wherein the light source is mounted in a cavity in one of the solid block portions and the light detector is mounted in a cavity in the other of the solid block portions.

15. The apparatus of claim 3, wherein the permeable membrane is made from a material selected from a group of membrane materials comprising mixed cellulose, ester, and polycarbonate.

16. A method for analysis of microbiological parameters in a fluid sample, comprising the steps of:
(a) providing a sterile sealed sample cuvette having a culture chamber and a detection chamber, the detection chamber being separated from the culture chamber by a permeable membrane, the permeable membrane having a pore size selected to block migration of a microbe under investigation from passing through the membrane into the detection chamber, each of the chambers having an inlet/outlet port for introduction, filtration and diffusion of fluid within the chambers;
(b) pre-loading the culture camber with a reagent specific to the microbe under investigation, the reagent having a soluble optically active ingredient that provides a detectable parameter in the presence of the microbe;
(c) introducing a fluid sample into the culture chamber through the inlet/outlet port using a suction filtration device and allowing the reagent to dissolve within the fluid sample to mix with reagent to form a dissolved reagent mixture;
(d) enabling the fluid and the detectable parameter to migrate through the membrane into the detection chamber;
(e) placing the sample cuvette into a holder within a housing and enclosing the housing;
(f) incubating the sample by means of temperature control components mounted within the housing proximal to the holder for microbiological culturing; and
(g) detecting the microbiological parameter by means of spectrophotometer components mounted within the housing proximal to the holder for photometric detection of the microbiological parameter.

17. The method of claim 16, wherein the enabling step comprises drawing the fluid through the permeable membrane using a suction pump connected to the outlet port of the detection chamber.

18. A method for analysis of microbiological parameters in a fluid sample, comprising the steps of:
(a) providing a sterile sealed sample cuvette having a culture chamber and a detection chamber, the detection chamber being separated from the culture chamber by a permeable membrane, the permeable membrane having a pore size selected to block migration of a microbe under investigation from passing through the membrane into the detection chamber, each of the chambers having an inlet/outlet port for introduction, filtration and diffusion of fluid within the chambers;
(b) introducing a fluid sample into the culture chamber through the inlet/outlet port using a suction filtration device connected to the inlet/outlet port of the detection chamber;
(c) filtering the sample by drawing the fluid through the membrane into the detection chamber and discarding the fluid through the outlet of the detection chamber until a desired volume of the sample is filtered;
(d) introducing a reagent solution into the culture chamber, the reagent solution having a reagent specific to the microbe under investigation, the reagent having a soluble optically active ingredient that provides a detectable parameter in the presence of the microbe;
(e) placing the sample cuvette into a holder within a housing and enclosing the housing;
(f) incubating the sample by means of temperature control components mounted within the housing proximate the holder for microbiological culturing; and
(g) detecting the microbiological parameter by means of spectrophotometer components mounted within the housing proximal to the holder for photometric detection of the microbiological parameter.
